SEQUENCE LISTING (3 sequences)

SEQ ID NO: 1 - protein, 999 aa, Homo sapiens
MADKVRRQRPRRRVCWALVAVLLADLLALSDTLAVMSVDLGSESMKVAIVKPGVPMEIVLNKESRRKTPVIVTLKENERFFGDSAASMAIKNPKATLRYFQHLLGKQADNPHVALYQARFPEHELTFDPQRQTVHFQISSQLQFSPEEVLGMVLNYSRSLAEDFAEQPIKDAVITVPVFFNQAERRAVLQAARMAGLKVLQLINDNTATALSYGVFRRKDINTTAQNIMFYDMGSGSTVCTIVTYQMVKTKEAGMQPQLQIRGVGFDRTLGGLEMELRLRERLAGLFNEQRKGQRAKDVRENPRAMAKLLREANRLKTVLSANADHMAQIEGLMDDVDFKAKVTRVEFEELCADLFERVPGPVQQALQSAEMSLDEIEQVILVGGATRVPRVQEVLLKAVGKEELGKNINADEAAAMGAVYQAAALSKAFKVKPFVVRDAVVYPILVEFTREVEEEPGIHSLKHNKRVLFSRMGPYPQRKVITFNRYSHDFNFHINYGDLGFLGPEDLRVFGSQNLTTVKLKGVGDSFKKYPDYESKGIKAHFNLDESGVLSLDRVESVFETLVEDSAEEESTLTKLGNTISSLFGGGTTPDAKENGTDTVQEEEESPAEGSKDEPGEQVELKEEAEAPVEDGSQPPPPEPKGDATPEGEKATEKENGDKSEAQKPSEKAEAGPEGVAPAPEGEKKQKPARKRRMVEEIGVELVVLDLPDLPEDKLAQSVQKLQDLTLRDLEKQEREKAANSLEAFIFETQDKLYQPEYQEVSTEEQREEISGKLSAASTWLEDEGVGATTVMLKEKLAELRKLCQGLFFRVEERKKWPERLSALDNLLNHSSMFLKGARLIPEMDQIFTEVEMTTLEKVINETWAWKNATLAEQAKLPATEKPVLLSKDIEAKMMALDREVQYLLNKAKFTKPRPRPKDKNGTRAEPPLNASASDQGEKVIPPAGQTEDAEPISEPEKVETGSEPGDTEPLELGGPGAEPEQKEQSTGQKRPLKNDEL

SEQ ID NO: 2 - protein, 13 aa, Homo sapiens
LAVMSVDLGSESM

SEQ ID NO: 3 - protein, 14 aa, Artificial Sequence
  Description of Artificial Sequence Synthetic peptide
CLAVMSVDLGSESM

(19) United States
(12) Patent Application Publication
(10) Pub. No.: US 2004/0265926 A1
(43) Pub. Date: Dec. 30, 2004

(54) Bodily fluid markers of tissue hypoxia

(76) Inventor: Leong Ng, Leicester (GB)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/719,695

(22) Filed: Nov. 21, 2003

(30) Foreign Application Priority Data
Sep. 24, 2003 (GB) 0322390.6
Nov. 21, 2002 (GB) 0227179.9

(51) Int. Cl. G01N 33/567

(52) U.S. Cl. 435/7.21

(57) ABSTRACT

The invention provides a method and kit for detecting tissue hypoxia, which is a clinical syndrome indicative of heart disease in a subject, by detecting an increased level of Oxygen Related Protein 150 (ORP150) in a bodily fluid sample from a subject.

### BACKGROUND OF THE INVENTION

Heart disease affects millions of people worldwide and is the leading cause of death in the United States. Chronic heart failure (CHF) is a common clinical syndrome which is an increasingly important health care issue in industrialized societies with elderly populations. Hospitalization rates for heart disease have increased markedly over the last 20 years and CHF is associated with poor prognosis and quality of life. The direct costs of CHF account for approximately 1-2% of health care expenditures, the vast majority being related to hospital admissions.

Chronic heart failure is most often the result of left ventricular systolic dysfunction (LVSD). Screening studies from Glasgow (McDonagh, et al, Lancet 1997; 350: 829-8331) and Birmingham (Davies, et al, Lancet 2001; 358: 439-444) indicated prevalence of rates of definite LVSD of 2.9% and 1.8% respectively. In both studies, the condition was asymptomatic in half of the cases. The identification of patients with LVSD allows the prescription of appropriate therapy which for the individual patient improves quality of life and prognosis. Echocardiography is currently the most frequently used investigation for the diagnosis of LVSD and heart failure.

The pathophysiology of heart failure involves activation of many neurohormonal systems, including the catecholamine, renin-angiotensin, endothelin, atrial and brain natriuretic peptide systems. Some of these systems are activated in an adaptive fashion (the natriuretic peptide systems); others are maladaptive (endothelin, renin-angiotensin and catecholamine systems). An increased secretion of the natriuretic peptide hormones has been exploited as a means for diagnosis of CHF (McDonagh et al, Lancet 1998; 351:9-13; Hobbs, et al, Br Med J 2002; 324: 1498-1502). For detection of LVSD, brain natriuretic peptide (BNP) is a better diagnostic tool than N-terminal pro-atrial natriuretic peptide (N-ANP) (McDonagh et al, Lancet 1998; 351:9-13). In addition, another peptide derived from the precursor of BNP, namely N-terminal proBNP (N-BNP) is also a reasonable alternative for the identification of LVSD (Hobbs, et al, Br Med J 2002; 324: 1498-1502). In both cases, the negative predictive values of the tests are high, suggestive of their utility in the exclusion of CHF.

In many cases of CHF, the etiology is ischaemic heart disease, of which the main cause is atherosclerosis. Reduced cardiac output over a chronic period leads to tissue hypoperfusion and relative tissue hypoxia. Accordingly, detection of an indicator in the plasma that is induced and secreted when tissues are hypoxic would have great utility in the diagnosis and prognosis of heart disease, and have further utility in the monitoring of such diseases.

Recent work has revealed oxygen regulated protein (ORP150) as a marker of tissue hypoxia. Originally cloned from astrocytes subjected to hypoxia, human ORP150 is an endoplasmic reticulum (ER) associated protein with a deduced amino acid sequence of 999 residues which includes a C-terminal ER retention signal-like sequence suggesting that this protein resides in the ER (Kuwabara, et al, J Biol Chem 1996; 271: 5025-32; Ikeda, et al, Biochem Biophys Res Commun 1997; 230: 94-9; U.S. Pat. No. 5,948,637). ORP150 has recently been listed in conjunction with a number of proteins that were cloned and classified as a secreted proteins based on the identification of a signal peptide in the deduced amino acid sequence (U.S. application Ser. No. 2003/0069405). The fact that ORP150 has a signal peptide was recognized by Ikeda et al., and they state “the existence of a signal peptide at the N-terminus and the ER-retention signal-like sequence at the C-terminus suggests that ORP150 resides in the ER, consistent with the results of immunocytochemical analysis reported by Kuwabara et al” (U.S. Pat. No. 5,948,637; Kuwabara, et al, J Biol Chem 1996; 271: 5025-32).

Accordingly, new markers are needed for effective early detection of heart disease. A marker that could be easily detected in bodily fluids would be particularly useful and provide a significant advance in the development of a non-invasive, sensitive, and highly reliable point-of-care ‘bedside test’ for individuals at risk for heart disease.

### SUMMARY OF THE INVENTION

The disclosed invention is based on the finding that the levels of Oxygen Regulated Protein 150 (ORP150) in body fluid are raised in patients at increased risk for heart disease.

In a first aspect, the disclosed invention provides methods for determining an increased risk of heart failure in a subject by detecting an increased level of ORP 150 in a sample of bodily fluid obtained from the subject.

In a preferred embodiment, the level of at least one further marker indicative of heart failure, such as N-terminal pro-Brain Natiuretic Peptide (NT-proBNP) or Brain Natiuretic Peptide (BNP) is also measured.

The level of ORP150 alone or in combination with another marker is preferably determined by use of an immunoassay, and the bodily fluid is preferably plasma.

In a second aspect, the disclosed invention provides kits for detecting the relative amount of ORP150 in a bodily fluid obtained from the subject at home or in a doctor's office.

Other features and advantages will be appreciated based on the following detailed description and claims.

### BRIEF DESCRIPTION OF THE DRAWINGS

FIG. 1 is a standard curve for the ORP150 peptide competitive immunoassay. A patient's plasma extract (solid circles joined by solid line) was diluted in two fold steps, showing parallelism with the standard curve. Two patients' urine extracts were also diluted in two fold steps (hollow triangles joined by dotted lines), again demonstrating parallelism with the standard curve.

FIG. 2 is a graph showing the results of size exclusion chromatography with analysis of the fractions for ORP150. The points of elution of markers for 150 kD, 20 kD and 6.5 kD are indicated by arrows. Three peaks of immunoreactivity for ORP 50 are evident at 150, approximately 7 and approximately 3 kD.

FIGS. 3a and 3b are box plots of log transformed plasma N-BNP and ORP150 levels respectively in normal subjects, heart failure patients, and patients with myocardial infarction.

FIGS. 4a and 4b are box plots of log transformed plasma N-BNP and ORP150 levels respectively in normal subjects and heart failure patients of both gender.

FIGS. 5a and 5b are graphs showing the relationship of plasma N-BNP and ORP150 respectively with severity of heart failure (as judged by the NYHA class) in males and females.

FIG. 6 is a receiver operating characteristic (ROC) curve for diagnosis of heart failure, using N-BNP or ORP150 alone, and using the prognostic index derived from a logistic model with a combination of N-BNP and ORP150.

FIG. 7 is a graph showing the relationship of plasma N-BNP and ORP150 to Killip class in patients after myocardial infarction.

FIG. 8 is a graph showing the relationship of plasma N-BNP and ORP150 to left ventricular function as assessed by echocardiography in patients after myocardial infarction. Ventricular dysfunction is classified as normal, mild, moderate or severe impairment.

FIG. 9 is a box plot of the levels of N-BNP and ORP150 to the clinical outcome of death in patients after myocardial infarction.

FIG. 10 is a box plot of the levels of N-BNP and ORP150 to the clinical outcome of rehospitalization with heart failure in patients after myocardial infarction.

FIGS. 11a and 11b are graphs showing survival analysis of patients following myocardial infarction, stratifying patients as below or above the median value of plasma N-BNP or of ORP150 respectively.

FIG. 12 is a graph showing survival analysis of patients following myocardial infarction, stratifying patients as having both plasma levels of N-BNP and ORP150 below or above the median, and an intermediate group in which either peptide is above their respective medians.

FIGS. 13a and 13b are graphs showing a comparison of the levels of N-BNP and ORP150 to the clinical outcome of death in patients after unstable angina/Non-ST elevation myocardial infarction.

FIGS. 14a and 14b are graphs showing survival analysis of patients following unstable angina/Non-ST elevation myocardial infarction stratifying patients as below or above the median value of plasma N-BNP or of ORP 150.

FIG. 15 is a graph showing survival analysis of patients following unstable angina/Non-ST elevation myocardial infarction, stratifying patients as having both plasma levels of N-BNP and ORP150 below or above the median, and an intermediate group in which either peptide is above their respective medians.

FIG. 16 is the amino acid sequence of human ORP150.

FIG. 17 is a graph showing the plasma levels of ORP150 and BNP (Brain Natriuretic Peptide-32) in patients undergoing coronary balloon angioplasty.

### DETAILED DESCRIPTION OF THE INVENTION

1. Definitions

For convenience, before further description of the disclosed invention, certain terms employed in the specification, examples, and appended claims are provided here.

The singular forms “a”, “an”, and “the” include plural references unless the context clearly dictates otherwise.

“ANP” refers to atrial natriuretic peptide, the first described peptide in a family of hormones which regulate body fluid homeostasis (see. Brenner et al., Physiol Rev. 1990; 70: 665).

The term “antibody” as used herein refers to binding molecules including immunoglobulin molecules and immunologically active portions of immunoglobulin molecules, i.e., molecules that contain an antigen binding site that specifically bind an antigen. The immunoglobulin molecules useful in the invention can be of any class (e.g., IgG, IgE, IgM, IgD and IgA ) or subclass of immunoglobulin molecule. Antibodies includes, but are not limited to, polyclonal, monoclonal, bispecific, humanized and chimeric antibodies, single chain antibodies, Fab fragments and F(ab′) fragments, fragments produced by a Fab expression library, anti-idiotypic (anti-Id) antibodies, and epitope-binding fragments of any of the above. An antibody, or generally any molecule, “binds specifically” to an antigen (or other molecule) if the antibody binds preferentially to the antigen, and, e.g., has less than about 30%, preferably 20%, 10%, or 1% cross-reactivity with another molecule. Portions of antibodies include Fv and Fv′ portions.

The term “bodily fluid” includes all fluids obtained from a mammalian body, including, for example, blood, plasma, urine, lymph, gastric juices, bile, serum, saliva, sweat, and spinal and brain fluids. Furthermore, the bodily fluids may be either processed (e.g., serum) or unprocessed.

“CNP” refers to C-type natriuretic peptide. (Stingo et al., Am. J Physiol., 1992; 263,:H1318).

“Comprise” and “comprising” are used in the inclusive, open sense, meaning that additional elements may be included.

The terms “detection” and “detecting” as used herein refer to methods of screening, diagnosis, prognosis, risk assessment, or disease stage assessment.

The term “heart disease” as used herein refers to the inability of the heart to keep up with the demands on it and, specifically, failure of the heart to pump blood with normal efficiency. Heart disease may result from heart failure, chronic heart failure, coronary artery disease (also ischaemic heart disease) leading to heart attacks and heart muscle weakness, primary heart muscle weakness from viral infections or toxins such as prolonged alcohol exposure, heart valve disease causing heart muscle weakness due to too much leaking of blood or heart muscle stiffness from a blocked valve, and hypertension (high blood pressure). Rarer causes include hyperthyroidism (high thyroid hormone), vitamin deficiency, and excess amphetamine (“speed”) use. Other causes of heart disease may include ischaemic cardiomyopathy, dilated cardiomyopathy, hypertensive cardiomyopathy, restrictive cardiomyopathy, valvular disease, vascular disease, and myocardial infarction.

As used herein, an “immunoassay” is an assay that utilizes an antibody to specifically bind to a marker.

The term “marker level” as used herein refers to the amount of marker in a sample of bodily fluid or a mammalian subject and refers to units of concentration, mass, moles, volume, concentration or other measure indicating the amount of marker present in the sample.

As used herein, the term “natriuretic peptide” includes a native ANP, BNP, or CNP, portions of, variants of, or chimeras thereof.

“NT-proBNP” or “BNP” refers to cardiac derived peptide hormone that circulates in the blood and exerts potent cardiovascular and renal actions. Mature hBNP consists of a 32 amino acid peptide containing a 17 amino acid ring structure formed by two disulfide bonds.

The term “NYHA classification” refers to the New York Heart Association (NYHA) classification. This is a four-stage classification where:

| | |
|---|---|
| Class 1. | Patients exhibit symptoms only at exertion levels |
| Class 2. | Patients exhibit symptoms with ordinary exertion. |
| Class 3. | Patients exhibit symptoms with minimal exertion. |
| Class 4 | Patients exhibit symptoms at rest. |

“ORP150” or “ORP” as used herein refers to oxygen regulated protein 150 (ORP150) or a fragment of ORP150. The amino acid sequence for human ORP150 is provided in FIG. 16 (NCBI database Accession AAC50947, Accession NP_{—}006380). The term ORP150 also includes portions of, variants of, or allelic variants thereof. In the disclosed invention, a fragment of ORP150 is a fragment of the ORP150 protein which has an amino acid sequence which is unique to ORP150. The fragment may be as few as 6 amino acids, although it may be 7, 8, 9, 10, 11, 12, 13, 14, 15 or more amino acids. In one embodiment, the fragment comprises or consists of the sequence LAVMSVDLGSESM. The fragment may have a molecular weight in the range of from 6 to 8, 6.5 to 7.5, 6.7 to 7.4, 1 to 4, 1.5 to 3.5, or 1.8 to 3.3 kD. The molecular weight may be determined by means known to those skilled in the art such as gel electrophoresis or size exclusion chromatography.

A “subject” refers to a human or a non-human animal.

The term “tissue hypoxia” as used herein refers to a decrease in tissue or organ oxygen supply below normal levels. Decreased oxygen supply may be attributed to reduced oxygen utilization, transport or flow resulting from a decreased number of red blood cells, defective oxygenation in the lungs (i.e. low tension of oxygen, abnormal pulmonary function, airway obstruction, or right-to-left shunt in the heart), reduced ability of hemoglobin to release oxygen, arteriolar obstruction, vasoconstriction, impairment of venous outflow or decreased arterial inflow. Tissue hypoxia may be indicative of a disease or of disease progression including heart disease comprising heart failure, chronic heart failure, ischaemic heart disease or coronary artery disease, myocardial infarction and other acute coronary syndromes (e.g. non-ST elevation myocardial infarction and unstable angina), atherosclerosis, aortic aneurysm, stroke, peripheral vascular disease, and valvular disease. Other diseases or conditions may include lung disease, chronic lung disease, tissue injury leading to cell necrosis, and tumor growth.

“Urotensin” or “UTN” refers to urotensin II polypeptide or fragments thereof (GenBank Accession Number NM_{—}021995, NM_{—}006786, 095399, and CAB63148).

2. General

The invention provides methods for detecting tissue hypoxia by measuring the level of ORP150 alone or in combination with at least one other marker in a bodily fluid sample whereby an elevated level of ORP150 relative to the normal level is indicative of tissue hypoxia and heart disease. Originally characterized as an endoplasmic reticulum protein it is surprising that ORP150 is secreted and further that it is secreted at a quantifiable level that can be detected in bodily fluid and used for the diagnosis of tissue hypoxia and heart disease.

The invention may also be used to assess the stage or severity of tissue hypoxia indicative of heart disease. Antibody detection of ORP150 alone or in combination with other markers may be used to detect ranges of increased levels of ORP150 which may reflect more advanced stages of heart disease.

The disclosed methods may be used for assessment of risk of developing tissue hypoxia as a result of recurring myocardial infarction. Antibody detection of ORP150 alone or in combination with other markers may be used to detect ORP150 at elevated levels to predict myocardial infarction recurrence.

The disclosed methods may also be used to monitor the effect of therapy administered to a subject having tissue hypoxia. Changes in antibody detection of ORP150 alone or in combination with another marker may reflect a subject's response to treatment.

3. Methods for Detecting Tissue Hypoxia

In the disclosed invention, the measured level of ORP150 (and, where measured, other marker(s) indicative of heart disease) is compared with the normal level. The normal level may be the level of ORP150 (or a second marker) typically found in the bodily fluid of a subject free from tissue hypoxia. These normal levels may be determined from population studies of subjects free from tissue hypoxia or with a previously determined reference range for ORP150 in such mammalian subjects. In one embodiment, the normal level may be determined when the subject is stabilized or is not suffering from or is suffering from less severe tissue hypoxia. This allows the relative changes of the marker(s) in the subject to be determined. Such subjects may be matched for age and/or gender.

The levels of ORP150 may be measured from plasma or a bodily fluid sample, such as interstitial fluid, whole blood, urine, lymph, and saliva, although any other body fluid, such as serum, gastric juices, and bile may be used. Methods of obtaining a bodily fluid sample from a mammalian subject are known to those skilled in the art.

In the disclosed invention, the measured level of ORP150 (and, where measured, other marker(s) indicative of heart disease) is compared with a normal level. The normal level may be the level of ORP150 (or further marker) typically found in the bodily fluid which is indicative of the absence of heart disease. These normal values of the levels of ORP150 typically found in a sample of bodily fluid which is indicative of the absence of heart failure may range from 104-956 fmol/ml. Where measured, the normal value of N-BNP that is indicative of the absence of heart failure may range from 1-5.7 fmol/ml. Levels of ORP150 that are indicative of an increased risk of heart failure may range from 956 fmol/ml or more. Levels of N-BNP that are indicative of an increased risk of heart failure may range from 5.7 fmol/ml or more. These subjects may be matched for age and/or gender.

The immunoassay may be comprised of an antibody or portion thereof sufficient for binding specifically to ORP150. One antibody useful for detecting ORP150 may recognize the sequence LAVMSVDLGSESM. Other suitable antibodies are available commercially from Immuno-Biological Laboratories Co. Ltd, 1091-1 Naka, Fujioka-shi, Gunma, 375-0005, Japan.

As mentioned, other markers that can be used in detecting tissue hypoxia or heart disease may be a natriuretic peptide, such as brain natriuretic peptide (BNP) or N-terminal pro-brain natriuretic peptide (N-BNP). The release of stored proBNP (the intact precursor to the two circulating forms, BNP (the active peptide) and N-BNP (the inactive peptide)) from cardiac myocytes in the left ventricle and increased production of BNP is triggered by myocardial stretch, myocardial tension, and myocardial injury. ORP150 may be useful in combination with the natriuretic peptides (e.g. N-terminal proBrain natriuretic peptide or N-BNP) in assessing the prognosis of patients with heart disease; after myocardial infarction, the combination of peptides is useful in risk stratification of patients with respect to mortality.

In further embodiments, the second marker may be another natriuretic peptide, such as atrial natriuretic peptide (ANP) and/or its inactive form, N-terminal proANP (NTproANP) (Hall, Eur J Heart Fail, 2001, 3:395-397). In other embodiments, the second marker may be CNP which functions as a vasodilating and growth-inhibiting peptide (Suga et al., J Clin, Invest., 1992, 90:1145; Stingo et al., Am. J Physiol., 1992, 262:H308; Stingo et al., Am. J Physiol.,1992, 263:H1318; Koller et al., Science, 1991, 252:120). Other secondary markers that could be used to diagnose heart failure may include non-polypeptidic cardiac markers such as sphingolipid, sphingosine, sphingosine-l-phosphate, dihydrosphingosine and sphingosylphosphorylcholine (see U.S. Pat No. 6,534,322). Additionally, urotensin II, a cardiovascular peptide with homology to the hormone of teleosts (Ames et al., Nature 1999; 16: 282-286) may be used as a second marker. When measuring the levels of the above natriuretic peptides, non-natriuretic peptides, non-poplypeptidic cardiac markers, or urotensin II, corrections for age and gender may be necessary in order to improve the accuracy of diagnosis.

Antibodies binding to BNP and ANP can be obtained commercially. Examples of commercially available antibodies binding to BNP are rabbit anti-human BNP polyclonal antibody (Biodesign International), rabbit anti-BNP amino acids 1-20 polyclonal antibody (Biodesign International), anti-human BNP monoclonal antibody (Immundiagnostik), and rabbit anti-human BNP amino acids 1-10 polyclonal antibody (Immundiagnostik). Examples of commercially available antibodies binding to ANP are mouse anti-human ANP monoclonal antibody (Biodesign International), rabbit anti-human ANP monoclonal antibody (Biodesign International), mouse anti-human ANP monoclonal antibody (Chemicon), rabbit anti-human ANP amino acids 95-103 antibody (Immundiagnostik), rabbit anti-human ANP amino acids 99-126 antibody (Immundiagnostik), sheep anti-human ANP amino acids 99-126 antibody (Immundiagnostik), mouse anti-human ANP amino acids 99-126 monoclonal antibody (Immundiagnostik) and rabbit anti-human a-ANP polyclonal antibody (United States Biological). Examples of commercially available antibodies binding to CNP include rabbit anti-C-Type Natriuretic Peptide-22 (Phoenix Pharmaceuticals).

Antibodies binding to urotensin can be obtained commercially. Examples of commercially available antibodies binding to urotensin include anti-urotensin (Phoenix Pharmaceuticals), rabbit anti-urotensin (Biodesign International), rabbit anti-human urotensin (Immundiagnostik).

Depending on the assays used to diagnose heart disease (see below), the antibodies specific to the markers of heart disease may further comprise a label, e.g., a fluorescent, enzymatic, or magnetic label. In such embodiments, the antibody is said to be “directly labelled.” An antibody can also be “indirectly labelled,” i.e., the label is attached to the antibody through one or more other molecules, e.g., biotinstreptavidin. Alternatively, the antibody is not labelled, but is later contacted with a binding agent after the antibody is bound to a specific marker of heart disease. For example, there may be a “primary antibody” and a second antibody or “secondary antibody” that binds to the Fc portion of the first antibody. Labels may be linked, preferably covalently, to antibodies according to methods known in the art.

Further depending on the assays used to diagnose heart disease, antibodies may be linked to a solid surface. The solid surface can be selected from a variety of those known in the art including plastic tubes, beads, microtiter plates, latex particles, gold particles, magnetic particles, cellulose beads, agarose beads, paper, dipsticks, and the like. Methods for direct chemical coupling of antibodies, to the cell surface are known in the art, and may include, for example, coupling using glutaraldehyde or maleimide activated antibodies. Methods for chemical coupling using multiple step procedures include biotinylation, coupling of trinitrophenol (TNP) or digoxigenin using for example succinimide esters of these compounds. Biotinylation can be accomplished by, for example, the use of D-biotinyl-N-hydroxysuccinimide. Succinimide groups react effectively with amino groups at pH values above 7, and preferentially between about pH 8.0 and about pH 8.5. Biotinylation can be accomplished by, for example, treating the antibodies with dithiothreitol followed by the addition of biotin maleimide.

Antibodies are preferably contacted with the sample of bodily fluid obtained from a mammalian subject at least for a time sufficient for the antibody to bind to a marker used to diagnose heart disease. For example, an antibody may be contacted with the sample of bodily fluid for at least about 10 minutes, 30 minutes, 1 hour, 3 hours, 5 hours, 7 hours, 10 hours, 15 hours, or 1 day.

The markers measured in the disclosed methods may be detected using an immunoassay. In one embodiment, an immunoassay is performed by contacting a sample from a subject to be tested with an appropriate antibody under conditions such that immunospecific binding can occur if the marker is present, and detecting or measuring the amount of any immunospecific binding by the antibody. Any suitable immunoassay can be used, including, without limitation, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion, immunodiffusion assays, agglutination assays, complement-fixation assays, immunoradiometric assays, fluorescent immunoassays, and protein A immunoassays.

For example, a marker can be detected in a fluid sample by means of a two-step sandwich assay. In the first step, a capture reagent (e.g., an anti-marker antibody) is used to capture the marker. The capture reagent can optionally be immobilized on a solid phase. In the second step, a directly or indirectly labelled detection reagent is used to detect the captured marker. In one embodiment, the detection reagent is an antibody. In another embodiment, the detection reagent is a lectin. Any lectin can be used for this purpose that preferentially binds to the marker rather than to other proteins that share the antigenic determinant recognised by the antibody. In a preferred embodiment, the chosen lectin binds to the marker with at least 2-fold, 5-fold or 10-fold greater affinity than to other proteins that share the antigenic determinant recognised by the antibody. A lectin that is suitable for detecting a given marker can readily be identified by methods well known in the art, for instance upon testing one or more lectins enumerated in Table I on pages 158-159 of Sumar et al., Lectins as Indicators of Disease-Associated Glycoforms, In: Gabius H-J & Gabius S (eds.), 1993, Lectins and Glycobiology, at pp. 158-174.

In one embodiment, a lateral flow immunoassay device may be used in the ‘sandwich’ format wherein the presence of sufficient marker in a bodily fluid sample will cause the formation of a ‘sandwich’ interaction at the capture zone in the lateral flow assay. The capture zone as used herein may contain capture reagents such as antibody molecules, antigens, nucleic acids, lectins, and enzymes suitable for capturing ORP150 and other markers described herein. The device may also incorporate one or more luminescent labels suitable for capture in the capture zone, the extent of capture being determined by the presence of analyte. Suitable labels include fluorescent labels immobilized in polysterene microspheres. Microspheres may be coated with immunoglobulins to allow capture in the capture zone.

Other assays that may be used in the methods of the invention include, but are not limited to, flow-through devices.

In a flow-through assay, one reagent (usually an antibody) is immobilized to a defined area on a membrane surface. This membrane is then overlaid on an absorbent layer that acts as a reservoir to pump sample volume through the device. Following immobilization, the remainder of the protein-binding sites on the membrane are blocked to minimize nonspecific interactions. When the assay is used, a bodily fluid sample containing a marker specific to the antibody is added to the membrane and filters through the matrix, allowing the marker to bind to the immobilized antibody. In an optional second step (in embodiments wherein the first reactant is an antibody), a tagged secondary antibody (an enzyme conjugate, an antibody coupled to a colored latex particle, or an antibody incorporated into a colored colloid) may be added or released that reacts with captured marker to complete the sandwich. Alternatively, the secondary antibody can be mixed with the sample and added in a single step. If a marker is present, a colored spot develops on the surface of the membrane.

In another embodiment, the invention provides the use of ORP150 as a diagnostic marker to determine the stage or severity of tissue hypoxia and heart disease in a mammalian subject. ORP150 may be used in combination with a further marker indicative of heart disease. The further marker may be a natriuretic peptide, such as brain natriuretic peptide (BNP) or N-terminal probrain natriuretic peptide (N-BNP). Levels of ORP150 may be measured from a sample of bodily fluid, which may be plasma, by use of an immunoassay. A diagnosis or prognosis may be made based upon the result obtained compared to that obtained from a healthy individual or individuals.

In an additional embodiment, the invention provides the use of ORP150 for assessing the prognosis of patients with heart disease (e.g. ischaemic heart disease or acute coronary syndromes) especially after myocardial infarction, levels of the peptide are elevated in patients at risk of increased mortality or readmission with heart failure.

In a further embodiment, the invention provides a method for monitoring the effect of therapy administered to a mammalian subject having tissue hypoxia. In this method, ORP150 levels can be measured (from a sample of bodily fluid by immunoassay) prior to the commencement of therapy to establish a base level for the subject. During the course of treatment, ORP150 levels will be monitored for deviations from this base level to indicate whether there is an increase or decrease of hypoxia and hence whether the therapy is effective. An increased level of ORP150 indicates tissue hypoxia.

4. Kits

The invention also provides a kit for measuring ORP150 and other markers useful for detecting tissue hypoxia and heart disease in a mammalian subject. Such a kit may be useful for monitoring the effect of therapy administered to a mammalian subject having tissue hypoxia. Said kit may comprise instructions for taking a sample of body fluid from a mammalian subject and one or more reagents for measuring the level of ORP150 in the sample. The one or more reagents may comprise an antibody which binds specifically to ORP150 and optionally another antibody which binds a second marker of tissue hypoxia or heart disease.

In addition, such a kit may optionally comprise one or more of the following: (1) instructions for using the kit for detection of tissue hypoxia or for monitoring the effect of therapy administered to a mammalian subject having tissue hypoxia; (2) a labelled antibody or optionally, a labelled binding partner to the antibody; (3) a solid phase (such as a reagent strip) upon which each antibody is immobilized; and (4) a label or insert indicating regulatory approval for diagnostic, prognostic or therapeutic use or any combination thereof. If no labelled binding partner to each antibody is provided, each antibody itself can be labelled with a detectable marker, e.g., a chemiluminescent, enzymatic, fluorescent, or radioactive moiety. Additional antibodies to other markers of heart disease may be included in the kit.

### EXEMPLIFICATIONS

The invention, having been generally described, may be more readily understood by reference to the following examples, which are included merely for purposes of illustration of certain aspects and embodiments of the invention, and are not intended to limit the invention in any way.

### EXAMPLE 1

### Assay of ORP150 in Normal Subjects and Heart Failure Patients

Study Populations

120 heart failure patients were studied, all with echocardiographically confirmed left ventricular systolic dysfunction (left ventricular (LV) ejection fraction<45%). A further 373 patients with myocardial infarction were also recruited. Acute myocardial infarction was defined as presentation with at least two of three standard criteria, i.e. appropriate symptoms, acute ECG changes of infarction (ST elevation, new LBBB), and a rise in creatine kinase (CK) to at least twice the upper limit of normal, i.e.>400 IU/L. 177 of the myocardial infarction patients were also investigated with echocardiography, with systolic function graded as normal, mild, moderate or severe impairment. Age and gender matched normal controls with LV ejection fraction>50%, were recruited from the local community by advertisement. All subjects gave informed consent to participate in the study, which was approved by the local Ethics Committee.

End Points in Myocardial Infarction Patients

End-points were defined as all-cause mortality and cardiovascular morbidity (rehospitalization with heart failure) following discharge from the index hospitalization. Multivariate analysis for all endpoints other than death was performed after the censorship of those patients dying during follow up.

Blood Sampling and Plasma Extraction

In normal subjects and heart failure patients, 20 mls of peripheral venous blood was drawn into pre-chilled Na-EDTA (1.5 mg/ml blood) tubes containing 500 IU/ml aprotinin after a period of 15 min bed rest. In myocardial infarction patients, a single blood sample was taken between 72-96 hours after symptom onset. After centrifugation at 3000 rpm at 4° C. for 15 min, plasma was separated and stored at −70° C. until assay. Prior to assay, plasma was extracted on C₁₈ Sep-Pak (Waters) columns and dried on a centrifugal evaporator. Some urine specimens were also collected from patients with heart failure. These were also extracted on C₁₈ Sep-Pak (Waters) columns as above.

Assay of ORP150

A peptide corresponding to the N-terminal domain (amino acids 33-45) of the human ORP150 sequence (LAVMSVDLGSESM) (Ikeda, et al, Biochem Biophys Res Commun 1997; 230: 94-9) was synthesized in the MRC Toxicology Unit, University of Leicester. Amino acids 1-32 may represent a signal sequence for the protein and may not be present in the mature ORP150 protein. A rabbit was injected monthly with this peptide conjugated to keyhole limpet hemocyanin using maleimide coupling to a cysteine added to the C-terminal of the sequence. IgG from the sera was purified on protein A sepharose columns. The above peptide was also biotinylated using biotin-maleimide in buffer containing (in mmol/l) NaH₂PO₄ 100, EDTA 5, pH 7.0 for 2 hours. After quenching with excess cysteine, the tracer was purified on HPLC using an acetonitrile gradient. Alternatively, the above peptide could be synthesized with incorporation of a biotinylated amino acid at the C- or N-terminus and used as a tracer. Plasma extracts and standards were reconstituted with ILMA (immunoluminometric assay) buffer consisting of (in mmol/l) NaH₂PO₄ 1.5, Na₂HPO₄ 8, NaCl 140, EDTA 1 and (in g/l) bovine serum albumin 1, azide 0.1. ELISA plates were coated with 100 ng of anti-rabbit IgG (Sigma Chemical Co., Poole, UK) in 100 μl of 0.1 mol/l sodium bicarbonate buffer, pH 9.6. Wells were then blocked with 0.5% bovine serum albumin in bicarbonate buffer. A competitive immunoluminometric assay was set up by preincubating 200 ng of the IgG with standards or samples within the wells. After overnight incubation, 50 μl of the diluted biotinylated ORP peptide tracer (2 μl/ml of the stock solution or a total amount of 100-500 fmol) was added to the wells. Following another 24 h of incubation at 4° C., wells were washed 3 times with a wash buffer (NaH2PO4 1.5 mmol/l, Na₂HPO₄ 8 mmol/l, NaCl 340 mmol/l, Tween 0.5 g/l, sodium azide 0.1 g/l). Streptavidin labeled with methyl-acridinium ester (MAE) was synthesized as described (Ng et al, Clinical Science 2002; 102: 411-416). Wells were incubated for 2 h with 100 μl of ILMA containing streptavidin-MAE (5 million relative light units per well). Following further washes, chemiluminescence was detected by sequential injections of 100 μL of 0.1 M nitric acid (with H₂O₂) and then 100 μL of NaOH (with cetyl ammonium bromide) in a Dynatech MLX Luminometer. The lower limit of detection (defined as 3 times standard deviation at zero peptide concentration) was 9.8 fmol per tube or 98 fmol/ml of plasma extracted. Within assay coefficients of variation were 3.1, 4.3 and 5.9% for 2, 30, 500 fmol/tube respectively. There was no cross-reactivity with peptides previously demonstrated to be elevated in heart disease such as ANP, BNP, N terminal proBNP or CNP.

Assay N-BNP

The assay for N-terminal proBNP was based on the non-competitive N-terminal proBNP assay described by Karl, et al, Scand J Clin Lab Invest Suppl 1999; 230:177-181. Rabbit polyclonal antibodies were raised to the N-terminal (amino acids 1-12) and C-terminal (amino acids 65-76) of the human N-terminal proBNP. IgG from the sera was purified on protein A sepharose columns. The C-terminal directed antibody (0.5 μg in 100 μL for each ELISA plate well) served as the capture antibody. The N-terminal antibody was affinity purified and biotinylated. Aliquots (20 μL) of samples or N-BNP standards were incubated in the C-terminal antibody coated wells with the biotinylated antibody for 24 hours at 4° C. Following washes, streptavidin labeled with methyl-acridinium ester (streptavidin-MAE, 5×10⁶ relative light units/ml) (Ng et al, Clinical Science 2002; 102: 411-416) was added to each well. Plates were read on a Dynatech MLX Luminometer as previously described (Ng et al, Clinical Science 2002; 102: 411-416). The lower limit of detection was 5.7 fmol/ml of unextracted plasma. Within and between assay coefficients of variation were acceptable at 2.3% and 4.8% respectively. There was no cross-reactivity with ANP, BNP or CNP.

Size Exclusion Chromatography and Gel Electrophoresis of Plasma Extracts

Plasma extracts were fractionated by isocratic size exclusion chromatography on a 300×7.8 mm Bio-Sep SEC S2000 column (Phenomenex, Macclesfield, Cheshire, UK) using 50 mmol/l NaH₂PO₄ (pH 6.8) at a flow rate of 1 ml/min as the mobile phase. Standards used to establish molecular weights included IgG (150 kD), BSA (68 kD), ovalbumin (44 kD), soybean trypsin inhibitor (20 kD), aprotinin (6.5 kD) and tryptophan (204 D) (from Sigma Chemical Co, Poole, UK.). Fractions collected every 20 sec were dried on a centrifugal evaporator before assaying for ORP150 as above.

Statistical Analysis

Statistical analysis was performed using SPSS Version 11.0 (SPSS Inc, Chicago, Mich.). Data are presented as mean ± SEM or median (range) for data with non-Gaussian distribution, which were log transformed prior to analysis. For continuous variables, one-way analysis of variance (ANOVA) was used. The interaction of multiple independent variables was sought using the univariate General Linear Model procedure with least significant difference P values reported. Pearson correlation analysis was performed and box plots were constructed consisting of medians, boxes representing interquartile ranges and the whiskers representing the 2.5^{th} to the 97.5^{th} centile. P values below 0.05 were considered significant. Kaplan Meier survival analysis was used to examine the usefulness of peptide levels in risk stratification following myocardial infarction.

Performance of the ORP150 Assay

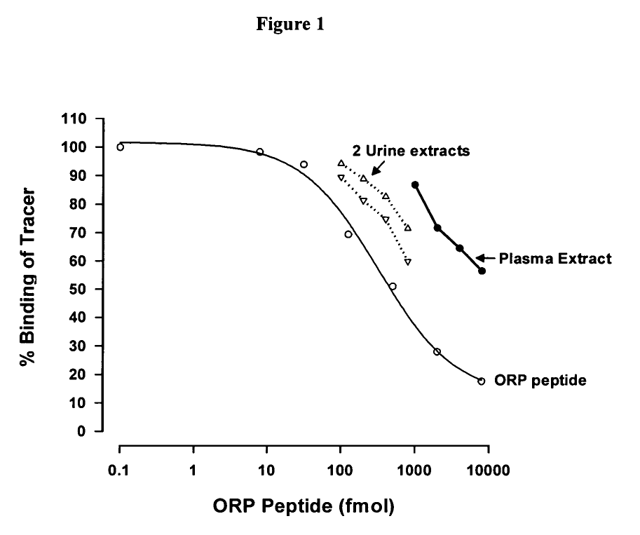

A typical standard curve for ORP150 peptide is illustrated in FIG. 1, showing a fall in chemiluminescence with increasing concentrations of the peptide. Half displacement of binding of the tracer occurred at about 300 fmol per tube. Dilutions of a heart failure patients' plasma and urine extracts showed parallelism with the standard curve. The lower limit of detection was 9.8 fmol/tube.

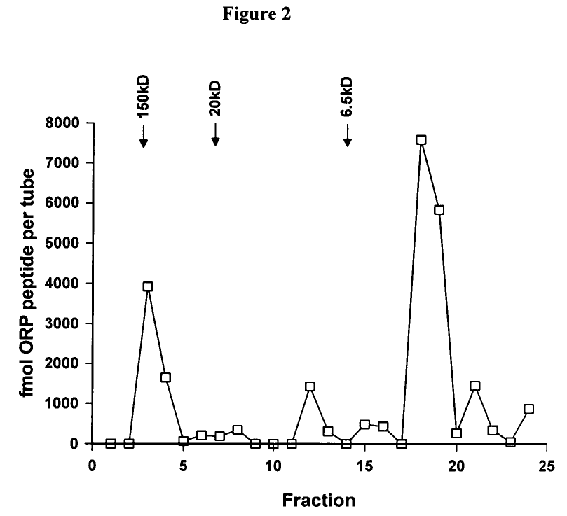

In addition, isocratic size exclusion chromatography was performed on human plasma extracts (FIG. 2). This was resolved into 3 main immunoreactive fractions, one at 150 kD (which is the expected molecular weight of human ORP150 protein), a smaller peak at 6.7 to 7.4 kD and the largest one at 1.8 to 3.3 kD. This suggests that ORP150 extracted from plasma is fragmented and there may be other fragments that could be detected with other epitope specific antibodies.

Conclusions on Detection of ORP in Humans

Specific immunoassays for ORP have detected the presence of this peptide in plasma and urine. As ORP150 is an endoplasmic reticulum associated protein, this finding is unexpected. Moreover, the immunoreactivity in plasma is derived from several molecular weight forms, suggesting that fragments of ORP150 may be detectable using epitope specific antibodies.

ORP150 in Normal Subjects, Heart Failure and Myocardial Infarction

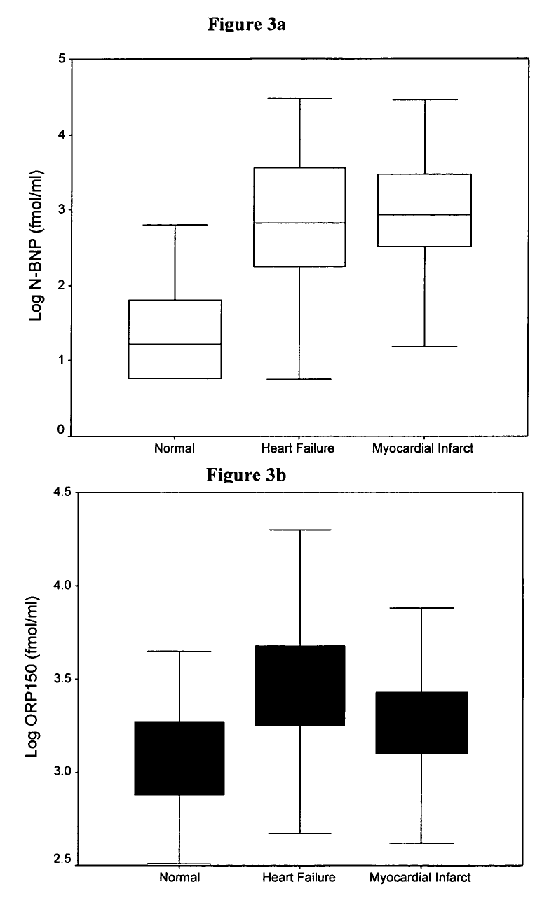

The characteristics of the normal, heart failure (HF) and myocardial infarction (MI) patients are shown in Table 1. Groups were well matched for gender. The normal and HF groups were matched for age, although the MI group was older than the other groups (P<0.001). Peptide levels were normalized by log transformation before analysis. FIG. 3 shows the N-BNP and the ORP150 levels in the normal, HF and MI patient groups. Using ANOVA, differences in Log N-BNP (P<0.0005) and Log ORP150 (P<0.0005) was evident between the 3 groups. For N-BNP, both the HF and MI patients' levels were higher than normal (P<0.0005 using Tukey's test for multiple comparisons), but levels in HF and MI groups were comparable (P not significant). For ORP150, both the HF and MI patients' levels were higher than normal (P<0.0005 using Tukey's test for multiple comparisons). Levels in the HF group were also significantly higher than those in the MI group (P<0.0005).

TABLE 1
Patient characteristics in the study. Means [ranges] are reported.

| | Normal | Heart failure | Myocardial |
|---|---|---|---|
| | Controls | patients | Infarction patients |
| Number | 180 (59 | 120 (35 (29%) | 373 (95 (26%) |
| | (32%)female) | female) | female) |
| Age (years) | 61.2 [26-81] | 61.4 [20-87] | 65.1 [32-95] |
| Drug therapy | | | |
| Diuretics | — | 98 | 176 |
| β blockers | — | 47 | 283 |
| ACE inhibitors | — | 99 | 220 |
| Etiology of | | | |
| Cardiomyopathy | | | |
| Ischaemic | — | 80 | 373 |
| Dilated | — | 29 | — |
| Hypertensive | — | 7 | — |
| Valvular | — | 4 | — |

ORP150 in Heartfailure

Within the normal group, there were age dependent changes in N-BNP (correlation coefficient r=0.438, P<0.0005). However, ORP150 was not significantly correlated with age. Combining the normal and HF groups, N-BNP was again correlated with age (r=0.306, P<0.0005) whereas the correlation of ORP150 with age was modest (r=0.138, P<0.02).

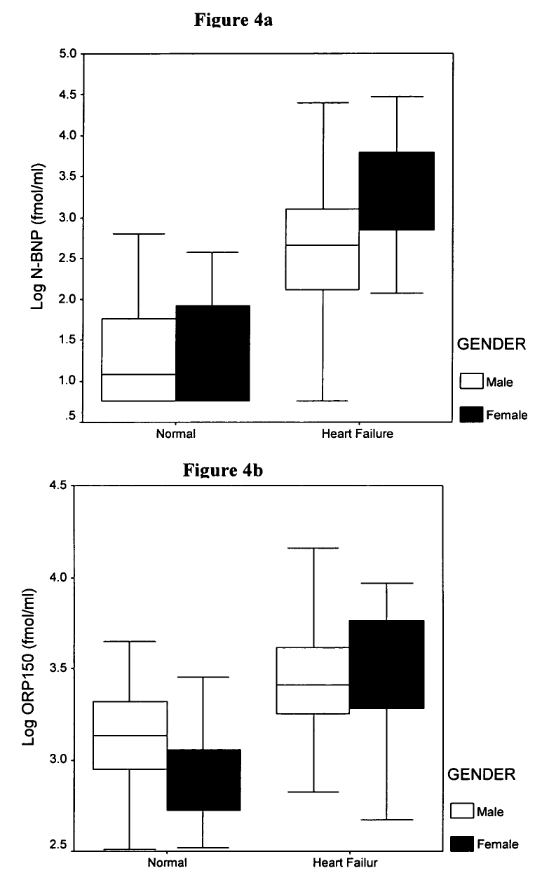

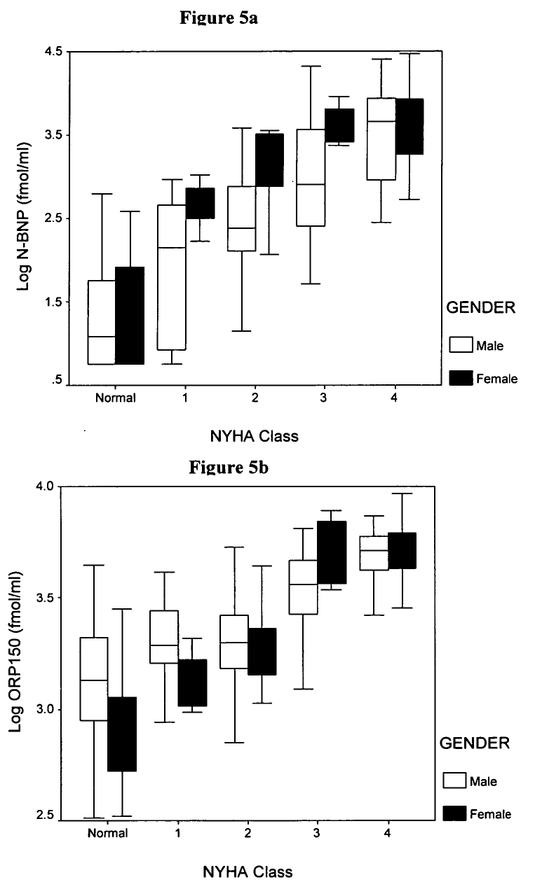

FIG. 4 shows the N-BNP and ORP150 levels in normal and HF subjects, for both gender. Levels of the peptides are elevated in both males and females with HF (P<0.0005 for both, using univariate general linear model (GLM) procedure). The elevation of both peptides in HF is dependent on the severity of HF as judged by the NYHA class. FIG. 5 shows that both peptides rise with increasing NYHA class in both gender. For N-BNP, values in normal subjects were different from NYHA class I, II, III and IV (P<0.0005 for all using Tukey's test). For ORP150, values in normal subjects were different from NYHA class I, II, III and IV (P<0.002, 0.0005, 0.0005, 0.0005 respectively using Tukey's test).

Using the univariate GLM procedure, and entering age as a covariate and gender and NYHA class as factors, analysis of the log normalized N-terminal proBNP levels in the heart failure patients yielded an r² of 0.675 for the model (P<0.0005) with age, gender and NYHA class as significant predictive variables (P<0.0005 for all). There was a significant interaction between gender and NYHA class, suggesting that the rise in N-BNP with increasing NYHA class may differ between males and females (P<0.007). A similar analysis performed on the log normalized ORP150 data yielded an r² of 0.512 for the model (P<0.0005) with NYHA class only as a significant predictive variable (P<0.0005). Age and gender were not significant predictive variables, although there was a significant interaction between gender and NYHA class (P<0.001) again suggesting that the rise in ORP150 with increasing NYHA class differs between males and females. Although the majority of HF patients have ischaemic heart disease as the aetiology, detection of HF using these peptides is achieved irrespective of aetiology.

For example, using a cut-off value of ORP150 of 956 fmol/ml, such a level based on the assay technique on plasma extracts described above would diagnose 95% of the HF cases, with a 39.4% specificity. ORP150 thus has a positive predictive value in this example, of 51.1% and a negative predictive value of 92.2%. Using such a cut-off value would enable effective exclusion of the diagnosis of HF.

A cut-off value such as this could be affected by assay methodology and different cut-off values need to established with new assays for ORP150, whether these are competitive or non-competitive assays, and whether peptide or protein standards are used (see note on assay methodology below).

Listed below are the cut-off values (in fmol/ml) for diagnosis of HF, for both N-BNP and ORP150, for a variety of sensitivities and the specificities are also reported.

TABLE II
Cut-off Values for N-BNP and ORP150
in fmol/ml for diagnosis of Heart Failure.

| | Cut-off | | |
|---|---|---|---|
| | Value | Sensitivity % | Specificity % |
| NBNP | 4.7 | 100 | 0 |
| | 5.7 | 95 | 40.6 |
| | 83.8 | 90 | 81.1 |
| | 118.3 | 85 | 87.2 |
| ORP150 | 104 | 100 | 0 |
| | 956 | 95 | 39.4 |
| | 1264 | 90 | 56.7 |
| | 1436 | 85 | 62.2 |

Stepwise logistic regression analysis was employed to predict absence or presence of HF, with log N-BNP and log ORP150 as predictive variables. Age and gender were not used since the normal and HF groups were age and gender matched. Both N-BNP (Odds ratio for 50% rise in peptide level 1.56, Odds ratio for 10 fold rise in peptide level 12.29, P<0.0005) and ORP150 (Odds ratio for 50% rise in peptide level 2.46, Odds ratio for 10 fold rise in peptide level 163.98, P<0.0005) were independent predictors of presence of HF, accounting for a total r² (Cox and Snell) of 0.55 and a Nagelkerke r² of 0.74 irrespective of whether forward or backward stepwise procedures was used.

Logistic regression involves fitting to the data an equation of the form ‘logit(p)=α+b₁x₁+b₂x₂+b₃x₃+. . .’, where logit(p)=logₑ (p/(1-p)), and p represents the probability of having HF, α is a constant and b₁ and b₂ represent coefficients which are multiplied by the variables x₁ and x₂ (in this example, x₁ and x₂ are logio (N-BNP) and log₁₀ (ORP150) ). This model could be used to calculate the probability of having heart failure, by measuring and then inputting the logio transformed N-BNP and ORP150 levels, logit(p)=−21.642+2.509*log₁₀(N-BNP)+5.1*log₁₀(ORP150).

Thus, if p is greater than 0.102, HF is detected with 95% sensitivity and 68.3% specificity. Note that this algorithm allows detection of heart failure with higher specificity than either of the peptides alone (at 95% sensitivity, specificities for N-BNP and ORP150 are only 40.6 and 39.4% respectively).

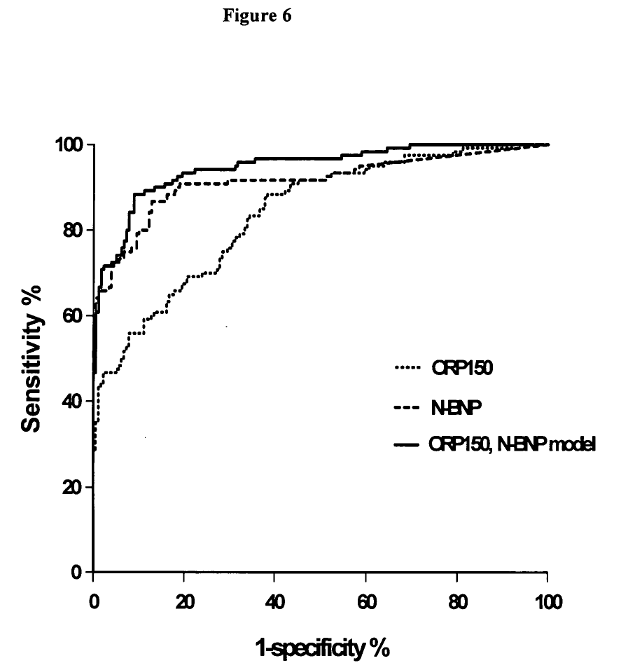

The prognostic index (probability of membership of HF group) derived from the above model was used to construct a receiver operating characteristic (ROC) curve (FIG. 6). The ROC area for the model was 0.95, greater than that of N-BNP (0.91) or ORP150 (0.84) alone, for the identification of HF.

The table below reports the sensitivity and specificity of the logistic model, using the logio transformed N-BNP and ORP150 levels, for various cut-off values of probability determined by the above algorithm. Different cut-off values of probability from the model could be picked depending on whether one wished to maximize the sensitivity of HF diagnosis, or its specificity.

TABLE III
Sensitivity and specificity of the logistic
model for cut-off values of probability.

| Cut-off Value of | | |
|---|---|---|
| probability in logistic | | |
| model | Sensitivity % | Specificity % |
| 0 | 100 | 0 |
| 0.102 | 95 | 68.3 |
| 0.313 | 90 | 84.4 |
| 0.483 | 85 | 91.1 |

Conclusions on ORP150 in Heart Failure

These findings suggest that although both N-BNP and ORP150 are elevated in HF (and with increasing severity of HF), N-BNP is more affected by age and gender of the subjects (with higher levels with rising age and in females). ORP150 by contrast does not have an age dependent component and is modestly affected by gender. Both peptides are effective in identification of HF, but the combination of the two may have added potential in diagnosis of HF.

ORP150 in Myocardial Infarction

The patient characteristics of the myocardial infarction (MI) group are shown in Table 1. Although gender matched were slightly older than the normal group (P<0.0005), both N-BNP and ORP150 were elevated in the plasma obtained 2-3 days after myocardial infarction (P<0.0005 for both, FIG. 3). Levels of N-BNP were correlated with the peak creatine kinase level (r=0.24, P<0.0005) suggesting a relation to the size of the infarction. However, ORP150 levels were not significantly correlated to the peak creatine kinase level (r=0.05, P not significant).

N-BNP was correlated to both age (r=0.39, P<0.0005) and creatinine (r=0.38, P<0.0005), the partial correlation coefficients remaining significant after allowing for the effects of gender and infarction (with age (r=0.39, P<0.0005) and with creatinine (r=0.36, P<0.0005)). In contrast, ORP150 was not significantly correlated with age, but weakly with creatinine (r=0.20, P<0.0005), the partial correlation coefficient falling further after allowing for the effects of gender and infarction (with creatinine (r=0.12, P<0.007).

The determinants of log normalized ORP150 were sought using stepwise linear regression analysis with age and creatinine as covariates, and presence of MI and gender as factors. Only presence of MI (P<0.0005) and creatinine (P<0.004) were identified as significant independent predictors of ORP150 levels, accounting for 14% of total variance (P<0.0005). A similar analysis with N-BNP levels identified age, gender, creatinine and presence of MI as significant independent predictors (P<0.0005 for all). Thus this finding confirms that in the HF group, i.e. ORP150 levels are less susceptible to influence by age and gender than N-BNP levels.

We used logistic regression analysis to predict presence or absence of MI as the dependent variable, using age, gender, N-BNP and ORP150 as independent variables. All 4 were identified as independent predictive variables for presence or absence of MI using both forward and backward stepwise regression analysis, the model accounting for an r² of 0.56 (Cox and Snell) or 0.79 (Nagelkerke). The odds ratios were as follows: for N-BNP (for a 50% rise in the peptide level 1.94, P<0.0005); for ORP150 (for a 50% rise in the peptide level 1.61, P<0.0005).

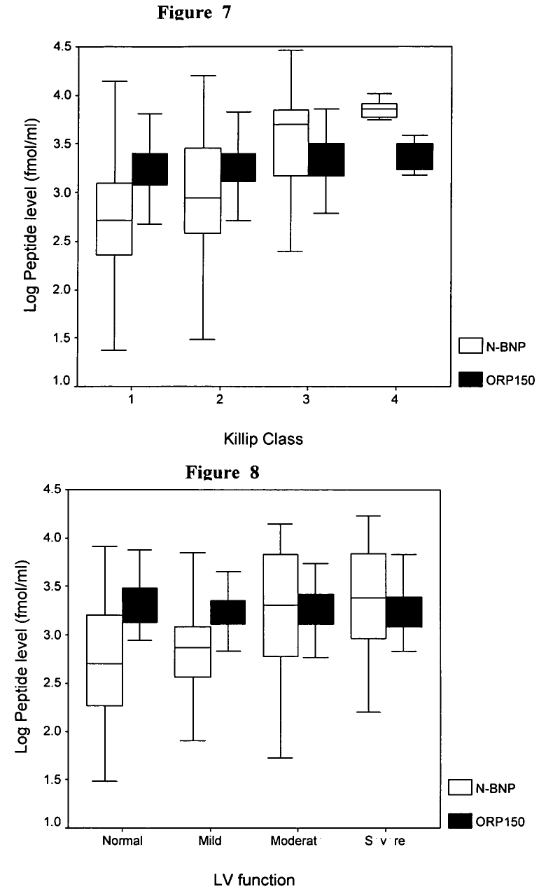

The plasma level of N-BNP was related to the Killip class of the patient (FIG. 7, P<0.0005). In contrast, levels of ORP150 were elevated in all MI patients irrespective of Killip class (FIG. 7). Of the 177 patients who had echocardiography scans, we found that the N-BNP levels was related to degree of LV dysfunction (FIG. 8, P<0.0005). In contrast, ORP150 levels were elevated in all MI patients irrespective of degree of LV dysfunction and even patients who had apparently “normal” LV function had elevated ORP150 levels (FIG. 8).

Outcomes after MI

All cause mortality and readmission rates with heart failure following MI were examined, to investigate the usefulness of ORP150 in prediction of these outcomes. Mean length of follow-up after discharge was 426 days with a range of 5-764 days. Out of the 367 cases, there were 39 deaths during the follow up period. There were also 22 readmissions with heart failure.

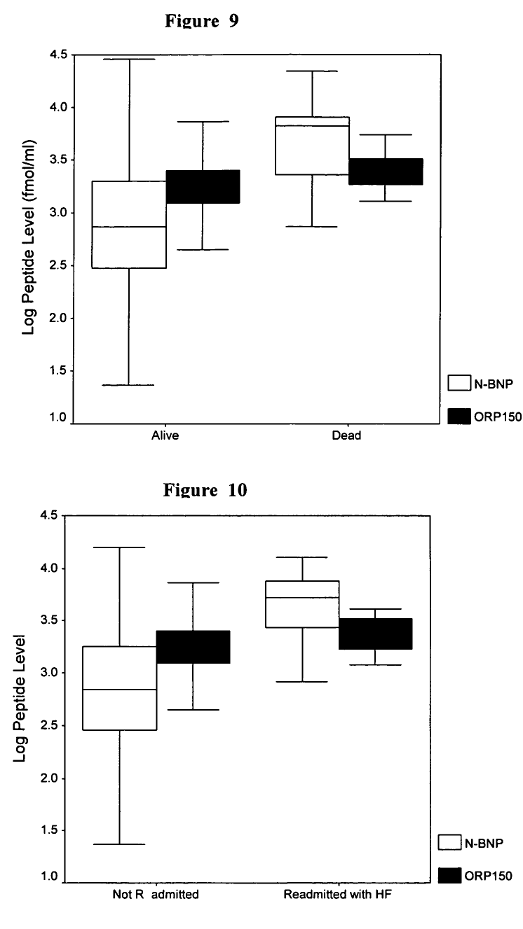

Patients who died had significantly higher N-BNP and ORP150 levels (P<0.0005 and P<0.001 respectively, FIG. 9). In addition, both peptides were elevated in those patients who were later readmitted with heart failure (P<0.0005 for N-BNP, P<0.025 for ORP150, FIG. 10).

Logistic regression analysis was used to investigate the predictors of death as an outcome with age, creatinine, past medical history of infarction, Killip class, and log N-BNP or Log ORP150. Significant independent predictors for death included N-BNP (odds ratio for 10 fold rise in peptide level 3.95, P<0.002) and ORP150 (odds ratio for 10 fold rise in peptide level 4.58, P<0.05), accounting for a Nagelkerke r² of 0.32. Backward and forward regression analysis confirmed these two independent predictor variables, but with an additional contribution from creatinine (odds ratio for 10 fold rise 19.56, P<0.05). These findings suggest that ORP150 is a predictor of mortality after MI independent of N-BNP levels.

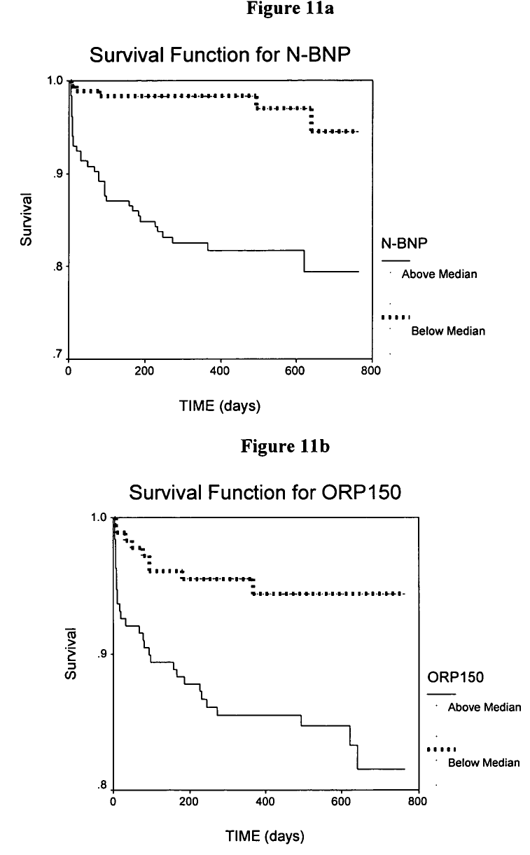

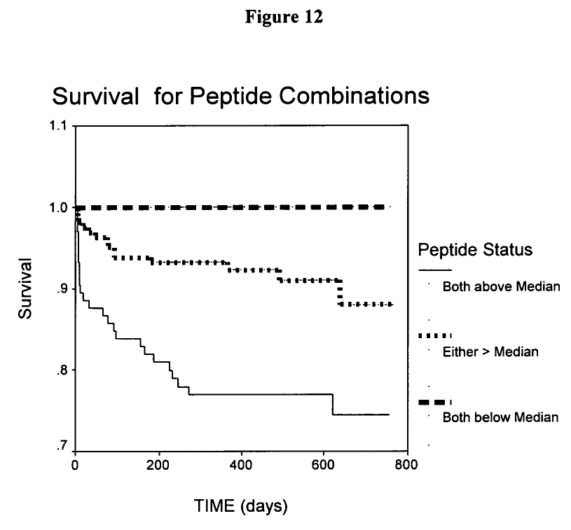

Kaplan Meier survival analysis was performed to confirm these findings. When subjects were divided in infra and supra median groups, survival differed significantly between these 2 groups (FIG. 11), whether the peptide used was N-BNP (P<0.0005 by log rank test for trend) or ORP150 (P<0.002 by log rank test for trend). Of note is that, even in the infra median groups defined by N-BNP or ORP150 alone, there is a definite mortality rate (albeit slower than the supra median groups). We utilized the ranks in both the N-BNP and ORP 150 ranked groups to yield a novel prognostic index, with patients divided up into 3 groups (both peptides below the medians, either peptide above the medians and both peptides above the medians). FIG. 12 shows the survival analysis using this new prognostic index, showing no deaths during the observational period in the group with both peptides below the medians, a high mortality rate in those with both peptides above the medians, and an intermediate mortality rate in those with either peptide above the medians (P<0.0005 by log rank test for trend).

The median ORP150 level for this particular example was 1820 fmol/ml, using the competitive assay technique on extracted samples as described above. For other assay formats using different standards, different median cut-off levels will need to be established (see below, note on methodology).

Conclusions on ORP in MI Plasma ORP150 levels are elevated in ischaemic heart disease as manifested by myocardial infarction. In contrast to N-BNP which is also elevated in these patients, ORP150 levels are less dependent on age, degree of LV dysfunction, symptoms and signs (as determined by Killip class) and renal function. Both peptides are good predictors of outcomes such as mortality or readmission with heart failure following the index admission with myocardial infarction. In particular, the combination of both peptides may be particularly useful in risk stratification after myocardial infarction (prediction of mortality).

Overall Conclusions on ORP in Vascular Disease

The above data demonstrates that ORP150 is secreted into human plasma and can also be found in urine. There may be fragments of ORP150 in bodily fluids. The levels of ORP150 are elevated in both Heart Failure and Ischaemic Heart Disease, and the measurement may be less prone to age and gender interference. As atherosclerosis is the major cause of vascular disease, ORP150 may be of use in the diagnosis or prognosis of other conditions where there is tissue hypoxia, for example, strokes, peripheral vascular disease, aneurysms, or acute coronary syndromes. In Heart Failure, in addition to being a diagnostic aid in itself, it could complement the measurement of N-BNP. In Myocardial Infarction, it may serve as an indicator of prognosis, predicting both death and readmissions with heart failure. Independently or in combination with N-BNP, its measurement after myocardial infarction is an effective aid to risk stratification able to detect extremely low or high risk groups of patients. This may have impact in the planning of therapeutic options for patients.

### EXAMPLE 2

### Assay for ORP150 in Unstable Angina/non-ST Elevation MI

A further 114 patients with unstable angina or non-ST elevation myocardial infarction (subendocardial myocardial infarction, defined as a rise of creatine kinase of under 2 fold upper limit of normal) were studied. All patients had chest pain at rest and were admitted to hospital for treatment. The mean (range) age was 66.8 years (38-93) and there were 74 men, 40 women. Blood samples were obtained at 3-5 days after admission to hospital, and analysed for troponin-T (Roche Diagnostics), ORP150 protein and N-BNP as detailed in Example 1.

Patients were followed up for end-points as described for myocardial infarction patients in Example 1.

During the mean follow up period of 401 days (range 26-764 days), there were 9 deaths. Troponin-T levels were not significantly different in those who died (0.12 (0.005-1.14) μg/L ) compared to those who survived (0.19 (0.005-0.557) μg/L).

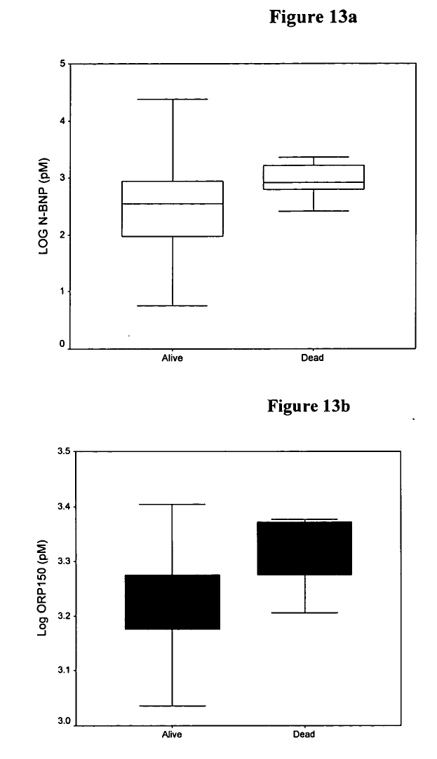

In contrast, both ORP150 and N-BNP levels were significantly higher in those who died compared to survivors (P<0.006 and P<0.05 respectively, FIG. 13).

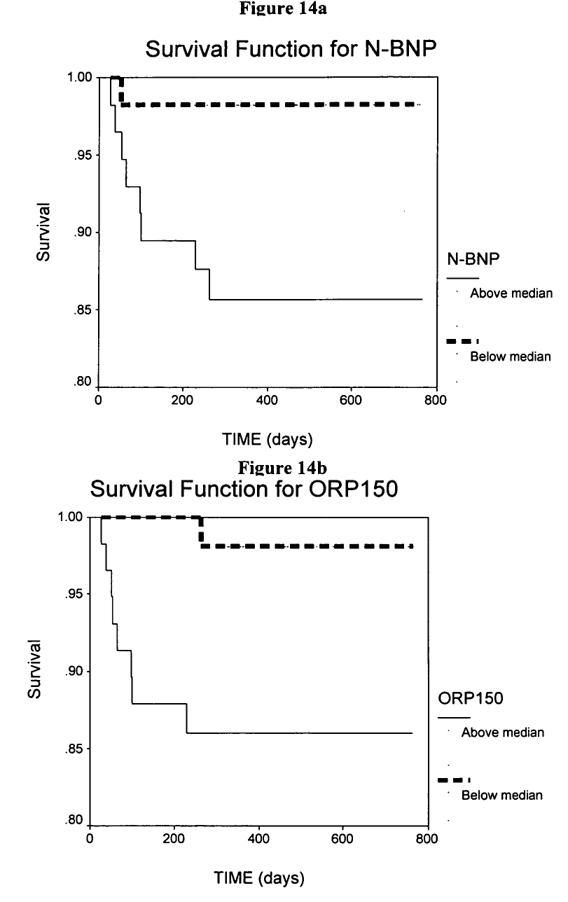

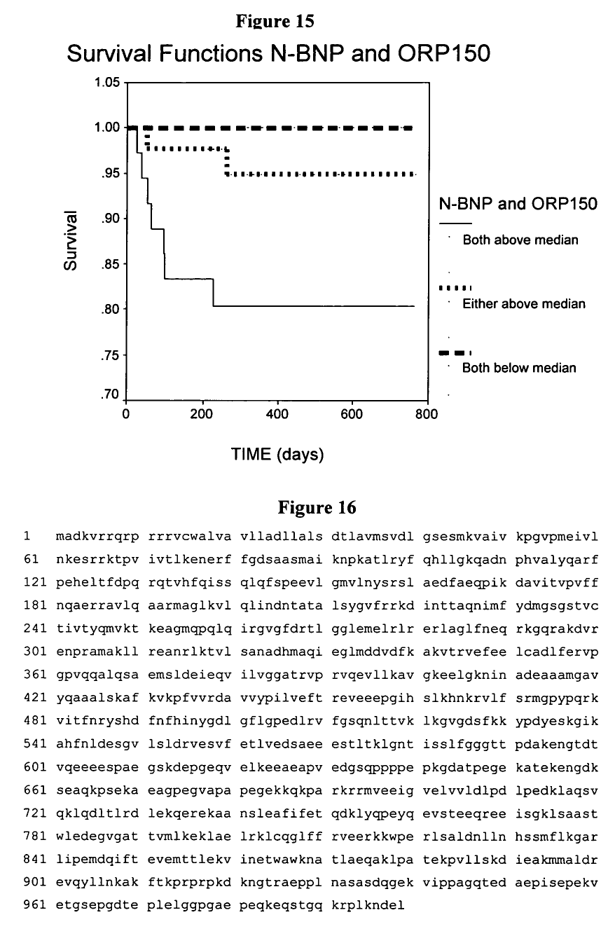

Kaplan Meier survival analysis was performed using both N-BNP and ORP150 levels (below or above median) for case stratification. When subjects were divided in infra and supra median groups, survival differed significantly between these 2 groups (FIG. 14), whether the peptide used was N-BNP (P<0.016 by log rank test for trend) or ORP150 (P<0.015 by log rank test for trend). When both peptides are used to classify patients into 3 groups (both peptides below the medians, either peptide above the medians and both peptides above the medians), the survival analysis suggested that those patients with both peptides below median had no deaths during the observational period, whereas those with both peptides above the median had a high mortality rate (P<0.002 by log rank test for trend, FIG. 15). This novel prognostic index for unstable angina/non-ST elevation myocardial infarction thus enables risk stratification similar to that described in the patients with ST-elevation myocardial infarction (as described above).

The median ORP150 level for this particular example was 1680 fmol/ml, using the competitive assay technique on extracted samples as described above. For other assay formats using different standards, different median cut-off levels will need to be established (see below, note on methodology).

Conclusions on ORP in Unstable Angina/Non-ST Elevation MI

Plasma ORP150 and N-BNP levels are elevated in ischaemic heart disease as manifested by unstable angina/Non-ST elevation MI. Both peptides are good predictors of outcomes such as mortality. In particular, the combination of both peptides may be particularly useful in risk stratification after unstable angina/Non-ST elevation MI (prediction of mortality). Use of such a prognostic index would enable treatment of the patients at highest risk of mortality with revascularization or pharmacological agents.

Note on Methodology to Establish ORP150 Cut-off Values in Examples

The cut-off values specified above are based on extracts of ORP150 from plasma, using peptide standards composed of CLAVMSVDLGSESM where LAVMSVDLGSESM is derived from the N-terminal sequence of ORP150. Due to the presence of the cysteine at the N-terminal (in order to produce the conjugates for immunisation in the first instance), there is a tendency for this peptide to form dimers. A variable proportion of dimers and monomers of the standard could lead to differences in immunoreactivity, and hence differences in actual cut-off values.

When an entire protein sequence is used as the standard (e.g. full length ORP150) or if the above peptide CLAVMSVDLGSESM is reduced using dithiothreitol and reacted with N-ethylmaleimide to prevent dimer formation, it is likely that immunoreactivity for this epitope with the antibodies raised could be different, and hence cut-off values could be different. Correction factors of up to 10-100 times the above mentioned cut-offs may need to be applied for different standards or different assay formats (e.g. a non-competitive as opposed to a competitive format). However, it is likely that cut-off values would lie in the range 10-10,000 fmol/ml and each new assay may have its own cut-off values assigned to it for each specific purpose (diagnosis or prognosis), in order to apply it to the uses described in the examples. These cut-off values will also differ according to whether the test is used for diagnosis of heart failure, or estimating prognosis after myocardial infarction or unstable angina, as illustrated in the examples above.

### EXAMPLE 3

Assay for ORP150 After Balloon Angioplasty

The effect of acute obstruction to the coronary circulation during balloon angioplasty was evaluated in 19 patients with coronary artery disease, who were undergoing this therapeutic procedure for treatment of atherosclerosis. Plasma was collected before the procedure, and at 2 hours, 6 hours and 12 hours after the angioplasty. The level of ORP150 was measured as described above. Additionally, the level of a known cardiac marker of ventricular wall stress which is known to be elevated after other coronary occlusion events such as myocardial infarction, namely B-type or Brain natriuretic peptide (BNP) was measured in C₁₈ column using an Immunoluminometric Assay.

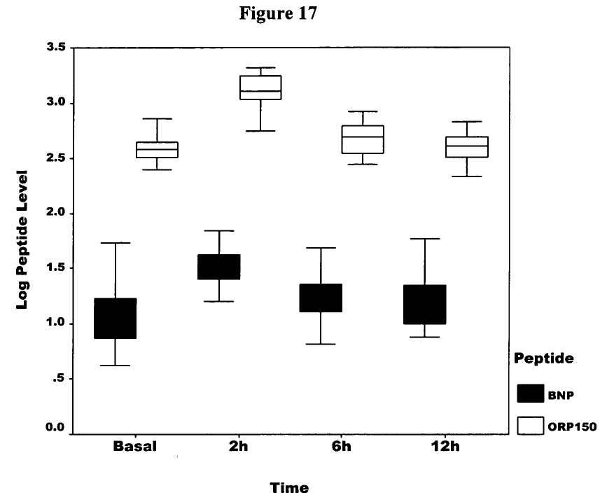

FIG. 17 illustrates the changes in plasma ORP150 levels after angioplasty compared to BNP. Both markers significantly change with time (P<0.001 using the analysis of variance with repeated measures). In addition, the plasma levels of both peptides peak at 2 hours after angioplasty, falling beyond that time back to baseline levels. Peak ORP150 levels at 2 hours were significantly different from basal (P<0.02) and 6 and 12 hour levels (P<0.001 for both). For BNP, peak levels at 2 hours were different from basal (P<0.001) and 6 and 12 hour levels (P<0.005 for both).

The rapid increase in ORP150 levels after balloon occlusion suggests that it can be used as an indicator of acute occlusion of the coronary circulation, as in myocardial infarction or other acute coronary syndromes (e.g. non ST elevation myocardial infarction or unstable angina).

Equivalents

The invention provides in part methods of detecting heart disease in a mammalian subject by measuring the levels of ORP150 in a sample of bodily fluid derived from a subject. While specific embodiments of the subject invention have been discussed, the above specification is illustrative and not restrictive. Many variations of the invention will become apparent to those skilled in the art upon review of this specification. The appendant claims are not intended to claim all such embodiments and variations, and the full scope of the invention should be determined by reference to the claims, along with their full scope of equivalents, and the specification, along with such variations.

All publications and patents mentioned herein are hereby incorporated by reference in their entireties as if each individual publication or patent was specifically and individually indicated to be incorporated by reference. In case of conflict, the present application, including any definitions herein, will control.

1. A method for detecting tissue hypoxia in a mammalian subject by detecting the level of oxygen related protein 150 (ORP150) in a bodily fluid sample whereby an elevated level of ORP150 relative to normal is indicative of an increased risk of heart disease.

2. The method of claim 1, wherein heart disease is the result of heart failure, chronic heart failure, coronary artery disease, ischaemic cardiomyopathy, myocardial infarction artherosclerosis, ischaemic stroke, aortic aneurysm, and peripheral vascular disease.

3. The method of claim 1, wherein the bodily fluid is plasma.

4. The method of claim 1, wherein the level of ORP150 is determined using an immunoassay.

5. The method of claim 1, wherein the immunoassay is a lateral flow immunoassay.

6. The method of claim 1, wherein the immunoassay is a flow-through immunoassay.

7. The method of claim 1, wherein the antibody is a monoclonal antibody.

8. The methods of claim 1, which comprises detecting a second marker whereby an elevated level of a second marker is indicative of an increased risk of heart disease.

9. The method of claim 8, wherein the second marker is a natriuretic peptide.

10. The method of claim 8, wherein the natriuretic peptide is brain natriuretic peptide (BNP) or N-terminal pro-brain natriuretic peptide (N-BNP).

11. The method of claim 8, wherein the level of second marker is determined by use of an immunoassay.

12. The method of claim 8, wherein the immunoassay is a lateral flow immunoassay.

13. The method of claim 8, wherein the immunoassay is a flow-through immunoassay.

14. The method of claim 8, wherein the bodily fluid is plasma.

15. The method of claim 8, wherein the mammalian subject is human.

16. The method of claim 8, wherein the level of ORP150 is monitored periodically.

17. The method of claim 8, wherein the level of a second marker is monitored periodically.

18. A kit for detecting an increased risk of tissue hypoxia and heart disease in a subject, comprising an antibody for detecting a level of ORP150 in a bodily fluid from a subject.

19. The kit of claim 18, further comprising an antibody for measuring the level of a second marker.

20. The kit of claim 18, wherein the second marker is N-BNP.

21. The kit of claim 18, wherein the second marker is BNP.
